(19) United States
(12) Patent Application Publication
(10) Pub. No.: US 2007/0219535 A1
(43) Pub. Date: Sep. 20, 2007

(54) Chest drainage anti-spill coupling

(76) Inventors: Trinh D. Phung, Attleboro, MA (US); Erich A. Dreyer, Bellingham, MA (US); Douglas W. Moore, Westwood, MA (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/377,670

(22) Filed: Mar. 17, 2006

(51) Int. Cl. A61M 27/00

(52) U.S. Cl. 604/541

(57) ABSTRACT

An anti-spillover flow coupling for a chest drainage assembly including at least a first and a second flow chamber. The coupling includes an intake port adapted to couple to the first flow chamber and exit port adapted to couple to the second flow chamber. The coupling defines a flow passageway between the intake port and exit port. The flow coupling further includes an anti-spill means for preventing the flow of liquid between the first flow chamber and second flow chamber. The anti-spill means can include a valve means including a ball member and valve seat member, the valve means being open when a valve axis is aligned substantially against the direction of gravity. Or the anti-spill means includes at least one filter element disposed over one of the intake port or exit port, the filter element permitting the passage of gases but not liquids.

### FIELD OF THE INVENTION

The present invention relates generally to medical devices. More particularly, the present invention relates to chest drainage units having blood collection chambers and suction and control elements for drawing fluids from a body.

### BACKGROUND OF THE INVENTION

Chest drainage devices and systems and more particularly suction drainage systems and devices for removing gases and/or liquids from medical patients, such as from the pleural cavity, by means of a pressure differential, are well known in the art. For many years, the standard apparatus for performing the evacuation of the pleural cavity was a drainage system known as the “3-bottle set-up” which includes a collection bottle, a water seal bottle, and a suction control bottle. A catheter runs from the patient's pleural cavity to the collection bottle, and the suction bottle is connected by a tube to a suction source. The three bottles are connected in series by various tubes to apply suction to the pleural cavity to withdraw fluid and air and thereafter discharge the same into the collection bottle. Gases entering the collection bottle bubble through water in the water seal bottle. The water in the water seal also usually prevents the back flow of air into the chest cavity. Suction or “negative” pressure is usually provided by a central vacuum supply in a hospital so as to permit withdrawal of fluids such as blood, water and gas from a patient's pleural cavity by establishing a pressure differential between the suction source and the internal pressure in the patient.

The 3-bottle set-up lost favor with the introduction of an underwater seal drainage system first sold under the name “Pleur-evac”® in 1966 by Deknatel Inc. U.S. Pat. Nos. 3,363,626; 3,363,627; 3,559,647; 3,683,913; 3,782,497; 4,258,824; and U.S. Pat. No. Re. 29,877 are directed to various aspects of the Pleur-evac® system, which over the years has provided improvements that eliminated various shortcomings of the 3-bottle set-up. These improvements have included the elimination of variations in the 3-bottle set-up that existed between different manufacturers, hospitals and hospital laboratories. A principal feature of the Pleur-evac® system is the use of a single, unitary, pre-formed, self-contained unit that embodies the 3-bottle techniques. Each unit generally includes a collection chamber for collecting body fluids drained from the pleural cavity of a patient. Such fluids can contain liquids such as blood or water. These liquids are meant to remain inside the collection chamber while a suction flow of gases travels from the collection chamber throughout the rest of the device and out through an attached suction line.

A common problem in known chest drainage devices occurs when the device is tipped over, causing fluids collected in the collection chamber to leak into the rest of the flow passageways in the device. This can occur fairly often when the unit is placed on the floor of a surgical room and similar such situations. The unit commonly also includes a water seal element as is well known in the art. When the unit is tipped over the fluids in the collection chamber and water seal chamber can mix, therein hampering further proper use of the device.

It is desirable therefore to provide for a way of preventing the spilling of collected fluids into other parts of a chest drainage device when such a device is tipped over. It is further desirable to maintain a proper flow passageway between various flow chambers inside a chest drainage device, while preventing the intermixing and flow of undesirable liquids such as water or blood, while allowing for the passage of suction gas flows and the transmission of suction pressures.

### SUMMARY OF THE INVENTION

In accordance with one embodiment of the present invention, an anti-spillover flow coupling is provided for a chest drainage assembly having an upright axis through an upright plane of orientation for the assembly. The assembly defines at least a first and a second flow chamber. The anti-spillover flow coupling includes an intake port adapted to couple to the first flow chamber and an exit port adapted to couple to the second flow chamber. The anti-spillover flow coupling includes a valve axis aligned in the direction of the upright axis of the chest drainage assembly when said intake port and exit port are coupled to the chest drainage assembly. A valve element is disposed in an interior space of the flow coupling between the intake port and exit port. The valve element has a ball member and defines a valve seat disposed above the ball member along the valve axis. The valve element defines, in an open position, a free flow passageway between the intake port and exit port to place the first and second flow chambers in fluid communication. The valve element is in the open position when the valve axis is aligned substantially opposite to the direction of gravity.

In accordance with another embodiment of the present invention, an anti-spillover flow coupling for a chest drainage assembly is provided, the assembly defining at least a first and a second flow chamber. The flow coupling includes an intake port adapted to couple to the first flow chamber, a first hydrophobic filter disposed over the intake port, an exit port adapted to couple to the second flow chamber, and a second hydrophobic filter disposed over the exit port. The flow coupling defines a gas flow passageway between the intake port and exit port to place the first and second flow chambers in fluid communication, the first hydrophobic filter impeding liquid flow from the first flow chamber into the gas flow passageway and the second hydrophobic filter impeding liquid flow from the second flow chamber into the gas flow passageway.

In accordance with yet another aspect of the present invention, an anti-spillover flow coupling for a chest drainage assembly is provided, the assembly defining at least a first and a second flow chamber. The anti-spillover flow coupling includes an intake port adapted to couple to the first flow chamber, and an exit port adapted to couple to the second flow chamber. The flow coupling defines a gas flow passageway between the intake port and exit port to place the first and second flow chambers in fluid communication, and includes an anti-spill means for preventing the flow of liquid between the first flow chamber and second flow chamber.

In accordance with still embodiment of the present invention, a modular chest drainage assembly with an upright axis is provided. The assembly includes a collection module which defines a collection chamber. The assembly further includes a flow control module defining at least one fluid flow pathway. An anti-spillover flow coupling is attachable to the collection module and flow control module to place the collection chamber and at least one fluid flow pathway in fluid communication. The anti-spillover flow coupling includes an anti-spill means for preventing the flow of liquid between the collection chamber and at least one fluid flow pathway.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a perspective view illustrating the separate components of a modular chest drainage device prior to final assembly, having a flow coupling according to one embodiment of the invention.

FIG. 2 is a top view of the assembly shown in FIG. 1.

FIG. 3 is a perspective cut-out view of a flow coupling according to an embodiment of the present invention.

FIG. 4A is a view of another flow coupling according to an embodiment of the present invention, prior to final assembly.

FIG. 4B is a view of the flow coupling of FIG. 4A, after filter elements have been attached to the coupling.

### DETAILED DESCRIPTION

The invention will now be described with reference to the drawing figures, in which like reference numerals refer to like parts throughout. An embodiment in accordance with the present invention provides a flow coupling with an anti-spill mechanism therein, configured to be coupled to a chest drainage assembly having an upright axis disposed through an upright plane of orientation for the assembly, where the assembly includes at least a first and a second flow chamber. The flow coupling provides a fluid flow pathway between the two chambers, where one chamber can be a collection chamber in a body fluid collection module and the second chamber can be a flow pathway in a flow control module. The anti-spillover flow coupling includes a valve element therein which is closed when the assembly unit is tipped over such that the upright plane forms an angle relative to gravity. Thus any fluid collected in the collection chamber is prevented from mixing with the pathways in the flow control module.

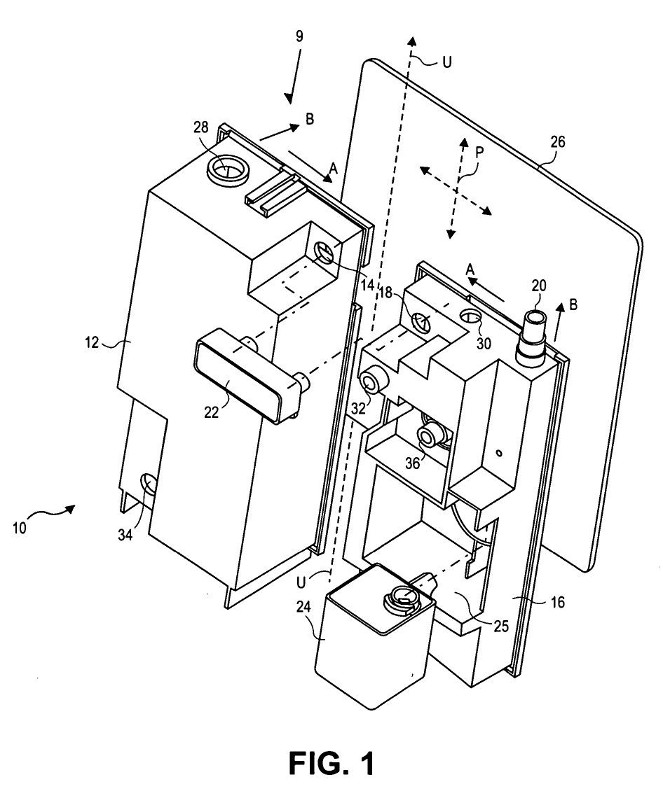

FIG. 1 is a perspective view illustrating the separate components of a modular chest drainage device prior to final assembly, having a flow coupling according to one embodiment of the invention. A modular chest drainage device 10 includes a collection module 12 defining a fluid collection chamber inside of it and having an exit port 14 for transmitting a suction flow out of the collection chamber. A ‘flow control’ module 16 defines an entry port 18 for receiving the suction flow from the collection chamber, and a suction port 20 for coupling to a suction source (not shown). An anti-spillover flow coupling 22 of the present invention is provided to be attached between the exit port 14 and the entry port 18, thereby bringing the collection module 12 and flow control module 16 in fluid communication. A pressure regulation module 24 is sealingly coupled to the pressure regulation flow intake port on the flow control module 16 and can be positioned in an enclosure 25 defined by the walls and geometry of the flow control module 16 as shown in FIG. 1. A face plate 26 is provided, wherein the collection module 12 and flow control module 16 are first aligned next to each other as in arrows A and then attached to face plate 26 as in arrows B so that the assembly can form multiple flow pathways, as will be illustrated in further detail below.

The collection module includes a fluid intake port 28 for receiving fluids from a patient. A catheter, tube, or similar device (not shown) can be coupled to the fluid intake port 28 in a variety of ways as is well known in the art. An ambient air port 30 is included on the flow control module 16 as part of a positive pressure relief valve element therein. A filling valve 32, such as a grommet or needle-less fill valve with a luer type fitting, is provided on the flow control module 16 for injecting fluids into the module for filling a manometer chamber or water seal chamber that is needed to control the backflow of gases and to indicate pressure, flow, or breathing, as further explained below. A re-infusion port 34 is provided on the collection module 12 for allowing collected body fluids to be returned to a patient by a re-infusion line. A high negativity pressure relief valve 36 is also provided on the flow control module 16 to prevent excessive negative pressures from building in the device.

Fluid entering the device 10 from a patient first passes through the fluid intake port 28 and enters a collection chamber defined inside the walls of the collection module 12. The collection chamber can be made up of any number of compartments or sub-compartments, as is well known in the art, and can vary in size depending on the nature of the patient body to which the chest drainage device is attached: i.e. adult vs. pediatric sizes. Suction pressures established throughout the device 10 are also present in the collection chamber such that gases entering the collection chamber are passed out of the chamber through exit port 14, while the liquid matter in the fluids captured inside of the collection chamber remains trapped inside the chamber. Suction pressure is thereby ‘transmitted’ such that a ‘suction flow’ is established between the intake port 28 and exit port 14, and further, via flow coupling 22, through the flow control module 16 and out through suction port 20. As used herein, the term ‘suction flow’ shall mean either a flow of gases or fluids from one point to another driven by a source of suction, or a flow in the direction of a negative pressure gradient, or an actual negative pressure gradient itself.

The overall assembly 10 is configured to be placed on a surface or attached or hung from a point such that an upright axis “U” as shown is substantially aligned with a gravity vector ‘g’, the direction of axis U pointing to the upwards or ‘upright’ orientation of the device 10. Upright axis U is coincident with an upright plane “P” which is coplanar with the span of face plate 26. Upright plane P thus generally denotes the plane of orientation of the major dimensional axes of the assembly, and is parallel to face plate 26.

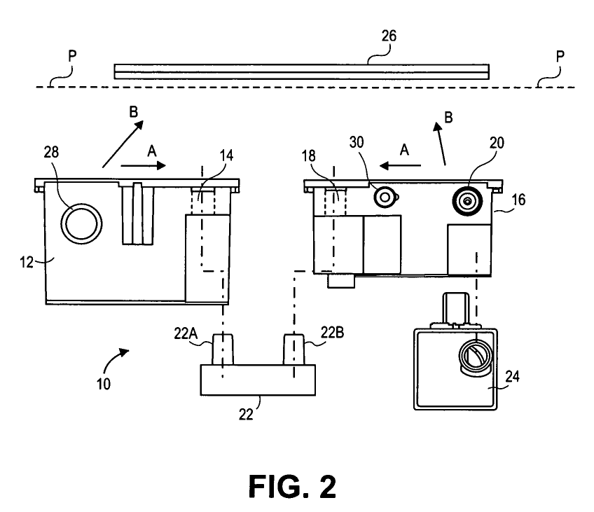

FIG. 2 is a top view of the assembly shown in FIG. 1. After the collection module 12 and flow control module 16 are aligned in the direction of arrows “A” to be positioned right next to one another, the flow coupling 22 is attached or coupled, permanently or detachably, to the collection module 12 through the flow exit port 14 with tubular extension 22a, and to the flow control module 16 through flow entry port 18 with tubular extension 22b. Thus, fluid or pressure communication, or a flow pathway, is established between the collection chamber inside of the collection module 12 and the flow pathways inside of flow control module 16.

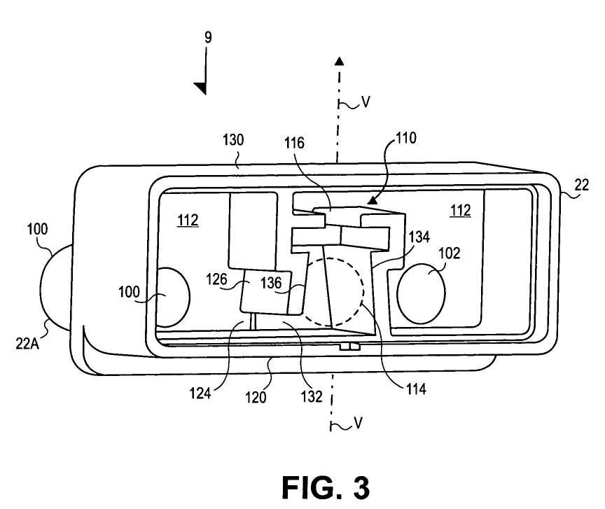

FIG. 3 is a perspective cut-out view of a flow coupling according to an embodiment of the present invention. Coupling 22 includes an intake port 100 adapted to couple to a first flow chamber in a chest drainage assembly. As used herein, the term “flow chamber” shall mean any space or pathway where fluids can collect or flow, such as a fluid collection chamber in collection module 12, or a flow pathway in flow control module 16. In the embodiment of FIG. 3, intake port 100 is configured to fit into and couple with exit port 14 on the collection module 12. The flow coupling 22 further includes an exit port 102 adapted to couple to a second flow chamber, this flow chamber being disposed inside the flow control module 16 via the entry port 18 defined therein. The anti-spillover flow coupling 22 has a valve axis “V” aligned in the direction of the upright axis U shown in FIG. 1, when the intake port 22a, 100, and exit port 22b, 102 are coupled to the chest drainage assembly 10.

The flow coupling 22 in FIG. 3 can further include a valve element or mechanism labeled generally as 110, disposed in an interior space 112 of the flow coupling 22 between the intake port 100 and exit port 102. The valve element 112 can include a spherical a ball member 114 (shown with dotted lines in FIG. 3) as well as a valve seat 116 disposed above the ball member 114 along the valve axis V. When in an open position, the ball member 114 rests near the bottom 120 of the flow coupling 22, such that a free flow passageway between the intake port 100 and exit port 102 is present through the interior space 112 to place the first and second flow chambers of the chest drainage assembly to which the coupling is attached in fluid communication. This free flow passageway runs: (i) from intake port 100, (ii) through a gap 124 defined by a rib element 126 inside the flow coupling 22, (iii) through the space shown in FIG. 3 surrounding the ball member 114 when said ball member is lying at near the bottom 120, (iv) through the open valve seat 116, and then out through exit port 102.

The flow coupling 22 includes a ‘bottom’ portion or edge 120 and a ‘top’ portion or edge 130, as oriented relative to the valve axis V. The valve element 110 functions to be in the open position when the valve axis vector V is aligned substantially opposite to the vector of the direction of gravity g as shown in FIG. 3. This is due to structure of the valve element 110 which defines a plurality of sloping surfaces 132, 134, 136 surrounding the ball member 114 below the valve seat 116, each of the sloping surfaces having a first end proximate the bottom 120 and a second end more proximate the valve seat 116 as shown in FIG. 3. An additional sloping surface is disposed on a backing (not shown due to cut-out view of FIG. 3) next to the ball member 114 so that the sloping surfaces surround the ball member 114 on four perpendicular sides. The surface 132 and that of the backing are oriented to diverge away from the valve axis V as they extend from the bottom 120 towards the valve seat. Thus, when the chest drainage assembly 10, and hence flow coupling 22, is tipped over on its face plate 26, such that the upright plane P, and hence valve axis V, makes an angle with the gravity vector, the ball member 114 is urged by gravity to roll along the surface 132 towards the valve seat 116 to thereby close the valve mechanism 110. This angle relative to the direction of gravity can vary at can be at least 90 degrees, or more, depending on whether a floor stand perpendicular to the upright plane is attached to the overall assembly 10 such that when the assembly is tipped over the face plate 26 forms an angle of more than 90 degrees with gravity. The sloping surface in the backing (not shown, directly opposite the surface 132 on the other side of the ball member 114) also diverges from the valve axis V as it extends towards the valve seat, such that if the assembly 10 were to be tipped over on its back surface opposite the face plate 26, the same gravity-driven closing mechanism would be triggered.

In addition, the sloping surfaces 134 and 136 that are perpendicular to diverging surface 132 are sloped to converge towards the valve axis V as they extend towards the valve seat 116, thereby guiding or urging the ball member 114 to abut against the valve seat 116 when the assembly is tipped over on its face plate 26 or against its back. Thus, the plurality of sloping surfaces around the ball member 114 cause the ball member 114 to be biased by to rest against the valve seat to block the free flow passageway. This prevents blood, water, or other fluids from being transferred between the two ports 100 and 102 when the chest drainage assembly is knocked over.

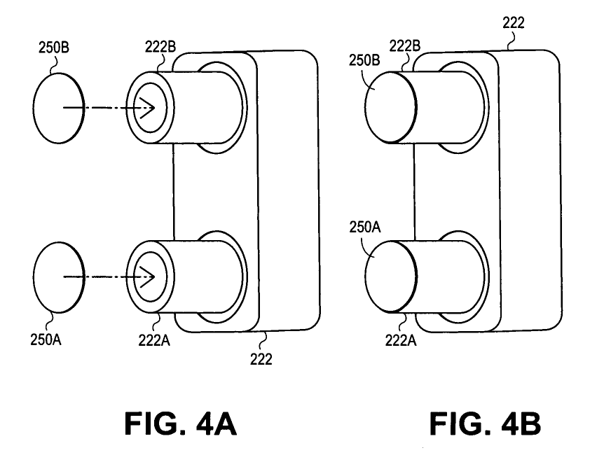

In addition to the gravity-driven anti spillover mechanism discussed and shown in FIG. 3, FIG. 4A is a view of another flow coupling according to an embodiment of the present invention, shown exploded prior to final assembly. Flow coupling 222 includes an intake port 222a and an exit port 222b similar to that of coupling 22 discussed in FIGS. 1-3. A first hydrophobic filter 250a is disposed over the intake port 222a, and a second hydrophobic filter 250b is disposed over the exit port 222b. FIG. 4B is a view of the flow coupling of FIG. 4A, after filter elements 250 have been attached to the coupling 222. The flow coupling 222 therefore defines a gas flow passageway between the intake port 222a and exit port 222b to place first and second flow chambers in a chest drainage assembly in fluid communication. The first hydrophobic filter 250a impedes liquid flow into the gas flow passageway in coupling 222 from the flow chamber to which intake port 222a is attached. And the second hydrophobic filter 250b impedes liquid flow into the gas flow passageway in coupling 222 from the flow chamber to which exit port 222b is attached. The filters 250 can be made of a vapor breathable material that repels water, like TYVEK™, or can possess the general property of being resistant to transmission of liquids such as water or blood, but permitting the passage of air or the transmission of pressure gradients or differentials, such that suction flow through the chest drainage assembly and flow coupling device is maintained.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. An anti-spillover flow coupling for a chest drainage assembly having an upright axis through an upright plane of orientation for the assembly, the assembly defining at least a first and a second flow chamber, the anti-spillover flow coupling comprising:
an intake port adapted to couple to the first flow chamber and an exit port adapted to couple to the second flow chamber, the anti-spillover flow coupling having a valve axis aligned in the direction of the upright axis of the chest drainage assembly when said intake port and exit port are coupled to the chest drainage assembly; and
a valve element disposed in an interior space of the flow coupling between the intake port and exit port, having a ball member and defining a valve seat disposed above the ball member along the valve axis, the valve element defining, in an open position, a free flow passageway between the intake port and exit port to place the first and second flow chambers in fluid communication, the valve element being in the open position when the valve axis is aligned substantially opposite to the direction of gravity.

2. The anti-spillover flow coupling of claim 1,
wherein the flow coupling includes a bottom and a top relative to the valve axis, and wherein the valve element further defines a plurality of sloping surfaces surrounding the ball member below the valve seat, each of the sloping surfaces having a first end proximate the bottom and a second end proximate the valve seat.

3. The anti-spillover flow coupling of claim 2,
wherein the valve element defines a closed position when the valve axis and upright plane of orientation of the assembly forms an angle relative to the direction of gravity of at least 90 degrees, wherein the ball member is biased by any of the plurality of sloping surfaces to rest against the valve seat to block the free flow passageway.

4. The anti-spillover flow coupling of claim 2,
wherein one of the plurality of sloping surfaces includes an opening defined by a rib inside the interior space by the flow coupling, the rib forming a channel through which the free flow passageway extends.

5. The anti-spillover flow coupling of claim 2,
wherein the plurality of sloping surfaces includes four sloping surfaces.

6. An anti-spillover flow coupling for a chest drainage assembly, the assembly defining at least a first and a second flow chamber, comprising:
an intake port adapted to couple to the first flow chamber;
a first hydrophobic filter disposed over the intake port;
an exit port adapted to couple to the second flow chamber;
a second hydrophobic filter disposed over the exit port;
the flow coupling defining a gas flow passageway between the intake port and exit port to place the first and second flow chambers in fluid communication, the first hydrophobic filter impeding liquid flow from the first flow chamber into the gas flow passageway and the second hydrophobic filter impeding liquid flow from the second flow chamber into the gas flow passageway.

7. The anti-spillover flow coupling of claim 6,
wherein the first and second hydrophobic filters permit passage of air but not liquid water.

8. The anti-spillover flow coupling of claim 6,
wherein the first and second hydrophobic filters permit passage of air but not blood.

9. The anti-spillover flow coupling of claim 6,
wherein the first hydrophobic filter permits passage of air but not liquid water.

10. The anti-spillover flow coupling of claim 6,
wherein the first hydrophobic filter permits passage of air but not blood.

11. An anti-spillover flow coupling for a chest drainage assembly, the assembly defining at least a first and a second flow chamber, the anti-spillover flow coupling comprising:
an intake port adapted to couple to the first flow chamber;
an exit port adapted to couple to the second flow chamber;
the flow coupling defining a gas flow passageway between the intake port and exit port to place the first and second flow chambers in fluid communication, and having an anti-spill means for preventing the flow of liquid between the first flow chamber and second flow chamber.

12. The anti-spillover flow coupling of claim 11,
wherein the anti-spill means includes a valve means including a ball member and valve seat member.

13. The anti-spillover flow coupling of claim 12,
wherein the flow coupling defines a valve axis along which the ball member and valve seat member are aligned, the valve seat member being above the ball member along the valve axis, the valve means being open when the valve axis is aligned substantially against the direction of gravity.

14. The anti-spillover flow coupling of claim 11,
wherein the anti-spill means includes at least one filter element disposed over one of the intake port and exit port, the filter element permitting the passage of gases but not liquids.

15. The anti-spillover flow coupling of claim 14,
wherein the at least one filter element prevents the passage of liquid water and blood.

16. A modular chest drainage assembly having an upright axis, comprising:
a collection module, the collection module defining a collection chamber;
a flow control module defining at least one fluid flow pathway;
an anti-spillover flow coupling attachable to the collection module and flow control module to place the collection chamber and at least one fluid flow pathway in fluid communication, the anti-spillover flow coupling having an anti-spill means for preventing the flow of liquid between the collection chamber and at least one fluid flow pathway.

17. The modular chest drainage assembly of claim 16,
wherein the anti-spill means prevents the flow of liquid between the collection chamber and at least one fluid flow pathway when the upright axis of the chest drainage assembly is non-aligned relative to the direction of gravity.

18. The modular chest drainage assembly of claim 16,
wherein the anti-spill means includes a valve means including a ball member and valve seat member.

19. The modular chest drainage assembly of claim 18,
wherein the flow coupling defines a valve axis along which the ball member and valve seat member are aligned, the valve axis being parallel to and in the direction of the upright axis, the valve seat member being above the ball member along the valve axis, the valve means being open when the valve axis is aligned substantially against the direction of gravity.

20. The modular chest drainage assembly of claim 16,
wherein the anti-spill means includes at least one filter element permitting the passage of gases but not liquids.
